(19) United States
(12) Patent Application Publication
(10) Pub. No.: US 2021/0315507 A1
(43) Pub. Date: Oct. 14, 2021

(54) SYSTEM AND METHODS FOR CONSCIOUSNESS EVALUATION IN NON-COMMUNICATING SUBJECTS

(71) Applicants: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITÉ, Paris (FR)

(72) Inventors: Federico RAIMONDO, Embourg (BE); Jacobo D SITT, Paris (FR); Lionel NACCACHE, Paris (FR); Diego FERNANDEZ SLEZAK, Buenos Aires (AR)

(73) Assignees: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITÉ, Paris (FR)

(21) Appl. No.: 17/272,482

(22) PCT Filed: Sep. 11, 2019
(86) PCT No.: PCT/EP2019/074231

§ 371 Date: Mar. 1, 2021

(30) Foreign Application Priority Data
Sep. 11, 2018 (EP) 18306190.2

(51) Int. Cl. A61B 5/377; A61B 5/349; A61B 5/0205; A61B 5/372; G06N 20/00

(52) U.S. Cl. CPC A61B 5/377; A61B 5/349; A61B 5/38; A61B 5/372; G06N 20/00; A61B 5/0205

(57) ABSTRACT

A method for the generation of a consciousness indicator for a non-communicating subject, including generating a sensory stimulation involving at least one of the five senses, the sensory stimulation having multiple consecutive stimuli including at least a first stimulus and a second stimulus relating to the same sense and different from one another, measuring an electrocardiographic signal of the subject during the generation of the sensory stimulation, extracting at least one EKG feature from the electrocardiographic signal associated to cognitive processes and generating a consciousness indicator using the EKG features as input of a classifier.

### FIELD OF INVENTION

The present invention pertains to the field of physiological signal evaluation. In particular, the invention relates to the reception and processing of at least one physiological signal in order to evaluate the state of consciousness of non-communicating subjects.

### BACKGROUND OF INVENTION

Patients with disorders of consciousness (DOC) are characterized by preserved wakefulness in the absence of clear evidence of awareness such that they remain unable to communicate with their surroundings.

For example, patients in a vegetative state/unresponsive wakefulness syndrome (VS/UWS) open their eyes, but they do not show conscious responses to sensory stimulation. When patients exhibit signs of fluctuating yet reproducible remnants of nonreflex behavior, such as visual pursuit, they are considered to be in a minimally conscious state (MCS). The diagnostic assessment of patients with disorders of consciousness is mainly based on the observation of motor and oculomotor behaviors at the bedside. The evaluation of nonreflex behavior, however, is not straightforward, as patients can fluctuate in terms of vigilance, and may suffer from cognitive and/or sensory impairments, from small or easily exhausted motor activity and pain, which may lead in the underestimation of the level of consciousness. Previous work employing data-driven analyses with neuroimaging and neurophysiological tools suggest relatively accurate patient diagnosis and prediction of clinical outcome. Most of this works uses measurements of brain electrophysiological activity by means of functional MRI or high density electroencephalograms (EEG).

However, neuroimaging, such as functional MRI and neurophysiological techniques are generally complex to implement, especially on patients that are in a vegetative state/unresponsive wakefulness syndrome or minimally conscious state. To perform neuroimaging, the patient usually needs to be moved from his bed to an imaging facility. When using neurophysiological techniques, the neurophysiological signal may be acquired on site without need to move the patient. However, due to level of accuracy needed to this task, the neurophysiological signal has to be acquired with high spatial density (i.e. high density EEG), producing a large amount of raw data which requires important calculation power. Therefore, data cannot be analyzed on site but need to be transfer for calculation to a remote device or server. Furthermore, neurophysiological techniques are sensible to electric signal of the order of the hundreds of μV in order to detect brain electric signal on the scalp of the subject and therefore are sensible as well to the electromagnetic noise.

In this context, the invention herein described proposes a solution allowing accurate patient diagnosis and prediction of clinical outcome through the analysis of physiological data that can be easily acquired on site, using physiological techniques that are noise robust and that produce a limited amount of data so that their analysis can be as well performed on site.

### SUMMARY

The present invention relates to a method for the generation of a consciousness indicator for a non-communicating subject, comprising:
generating a sensory stimulation involving at least one of the five senses, said sensory stimulation comprising multiple consecutive stimuli including at least a first stimulus and a second stimulus relating to the same sense and different from one another;
measuring an electrocardiographic signal of the subject obtained from a recording during the generation of the sensory stimulation;
extracting at least one EKG feature from the electrocardiographic signal associated to cognitive processes; and
generating a consciousness indicator by using the EKG features as input of a classifier.

The method of the present invention advantageously allows the use of an electrocardiographic signal to deduce a consciousness indicator for state evaluation of non-communicating subject. Electrocardiogram is a technique of simple implementation in the medical environment, requiring a limited number of electrodes (ranging from 2 to 12), notably inferior to the number of electrodes implemented in high density EEG (ranging from 64 to 256). Therefore, the amount of raw data to be analyzed is significantly reduced, which requires low computing power which is compatible to the capability of any conventional computer. Furthermore, cardiac electrical signal is of the order of the mV, providing signal to noise ratio of electrocardiographic signal much higher than the one obtainable for EEG. Using electrocardiographic signal obtained directly from electrocardiogram instead of cardiac signal estimation extracted from other measurement technics (inertial motion sensors or sounds) or other physiological signal measurement (electroencephalographic signal) has the advantage of comprising more meaningful information resulting in more EKG features that can extracted.

According to one embodiment, the stimuli of the sensory stimulation are auditory stimuli, visual stimuli, somatosensory stimuli, olfactory stimuli, gustatory stimuli, or a combination thereof.

According to one embodiment, the extraction step comprises the steps of:
performing a delineation of the EKG electrocardiographic signal identifying for each heart beat a QRS complex, a T-wave and a P-wave;
calculating the heart rate and heart rate variability for the electrocardiographic signal from the delineation of the EKG electrocardiographic signal;
defining a time interval as the interval between the onset of at least one stimulus and the following R peak and extracting one EKG feature calculating a modulation of the correlation of the phase of the cardiac cycle and the timing of the sensory stimulation.

According to one embodiment, the present method further comprises a step of receiving at least one additional physiological data of the subject recorded during the sensory stimulation chosen among the following: respiratory activity measurements, electrodermal activity measurements, metabolic parameter measurements, pupillometry measurements.

According to one embodiment, the extraction step further comprises extracting at least one physiological feature, notably by calculation of a modulation of the correlation of the physiological signal and the timing of the sensory stimulation.

According to one embodiment, the extraction step further comprises extracting at least one physiological feature, notably by calculating a phase shift of the physiological signal.

According to one embodiment, the at least one physiological feature is used as further input of the classifier.

Including further information concerning cognitive processes using the physiological features advantageously improves performances of the classifier. The use of features extracted from physiological signal is particular advantageous since physiological signals are simple to acquire, produce a reduced amount of raw data and therefore demand low computing power.

According to one embodiment, the method of the present invention further comprises a step of measuring an electroencephalographic signal of the subject during the generation of the sensory stimulation.

According to one embodiment, the method of the present invention further comprises the steps of:
extracting at least one EEG feature from the electroencephalographic signal; and
using the EEG feature(s) as further input of the classifier to generate a consciousness indicator.

Use of EEG extracted features has the advantage of further improving the performances of the classifier.

According to one embodiment, the classifier is a previously trained machine learning technique.

According to one embodiment, the method of the present invention comprises a step of comparison of the consciousness indicator to a predefined threshold.

The present invention further relates to a program comprising instructions which, when the program is executed by a computer, cause the computer to carry out the steps of the method according to any one of the embodiments described hereabove.

The present invention further relates to a non-transitory computer readable medium comprising instructions which, when the program is executed by a computer, cause the computer to carry out the steps of the method according to any one of the embodiments described hereabove.

Yet another aspect of the present invention relates to a system for the generation of a consciousness indicator for a non-communicating subject, comprising:
an acquisition module configured to acquire an electrocardiographic signal of the subject during the generation of the sensory stimulation; and
a calculation module configured to extract at least one EKG feature from the electrocardiographic signal and deduce a consciousness indicator using the EKG feature as input of a classifier.

According to one embodiment, the system further comprises a stimuli generator configured to generate sensory stimulation, said sensory stimulation comprising multiple consecutive stimuli including at least a first stimulus and a second stimulus relating to the same sense and different from one another;

According to one embodiment, at least the acquisition module and the calculation module are comprised in a same portable element configured to locally implement the method according to any one of the embodiments described hereabove.

The low computing power required for the method implementation allows advantageously to include the acquisition module and the calculation module in a portable element which is easily displaceable, notably to be taken directly in proximity to the subject in a medical facility.

### DEFINITIONS

In the present invention, the following terms have the following meanings:
“Electrode” refers to a conductor used to establish electrical contact with a nonmetallic part of a circuit, preferably a subject body. For instance, EEG electrodes are small metal discs usually made of stainless steel, tin, gold, silver covered with a silver chloride coating; there are placed on the scalp at specific positions.
“Electrocardiographic signal” refers to the signal recording the electrical conduction in the heart. Said cardiac signal may be for instance an electrocardiogram (EKG or ECG) or an endocardiogram. Such signals may have one or more channels, called leads. It may be short term (10 seconds in standard EKGs) or long term (several days in Holters).
“Electroencephalogram” or “EEG” refers to the tracing of brain waves, by recording the electrical activity of the brain from the scalp, made by an electroencephalograph.
“Electroencephalograph” refers to an apparatus for amplifying and recording brain waves.
“Heart rate variability (HRV)” refers to the physiological phenomenon of variation in the time interval between heartbeats. It is measured by the variation in the beat-to-beat interval.
“Mismatch negativity (MMN)” refers to a brain response to violations of a rule, established by a sequence of sensory stimuli (typically in the auditory domain). The mismatch negativity reflects the brain's ability to perform automatic comparisons between consecutive stimuli and provides an electrophysiological index of sensory learning and perceptual accuracy.
“P300 wave” refers to event related potential (ERP) component elicited in the process of decision making. More specifically, the P300 is thought to reflect processes involved in stimulus evaluation or categorization.
“Subject” refers to a mammal, preferably a human. In the sense of the present invention, a subject may be a patient, i.e. a person receiving medical attention, undergoing or having underwent a medical treatment, or monitored for the development of a disease.

### BRIEF DESCRIPTION OF THE DRAWINGS

Features and advantages of the invention will become apparent from the following description of embodiments of a method and a system for the generation of a consciousness indicator for non-communicating subjects according to the invention, this description being given merely by way of example and with reference to the appended drawings in which:

FIG. 1 is a flowchart showing the main steps of the method according to the present invention.

FIG. 2 is an illustration of the local global auditory paradigm according to one embodiment of the present invention.

FIG. 3 is an illustration of a RR interval in an EKG signal and time interval between the onset of the N^{th} sensory stimulus and the following R peak.

FIG. 4 is a flowchart showing the steps implemented by the method according to a second embodiment of the invention concerning the classification of subjects with disorders of consciousness using EKG features and physiological features.

FIG. 5 is a flowchart showing the steps implemented by the method according to a third embodiment of the invention concerning the classification of subjects with disorders of consciousness using EKG features, EEG features and physiological features.

FIG. 6 is a flowchart showing the steps implemented by the method according to a fourth embodiment of the invention concerning the classification of subjects with disorders of consciousness using EKG features and EEG features.

FIG. 7 is a schematic representation of the system according to the one embodiment.

### DETAILED DESCRIPTION

This invention relates to a method for characterizing the consciousness state of subjects with disorders of consciousness. To this end, the method is configured to receive and analyze at least cardiac activity data, comprising information linked to cognitive process in the subject, so to generate a consciousness indicator. Indeed, cardiac activity is a peripheral body signal that is linked to cognitive processes.

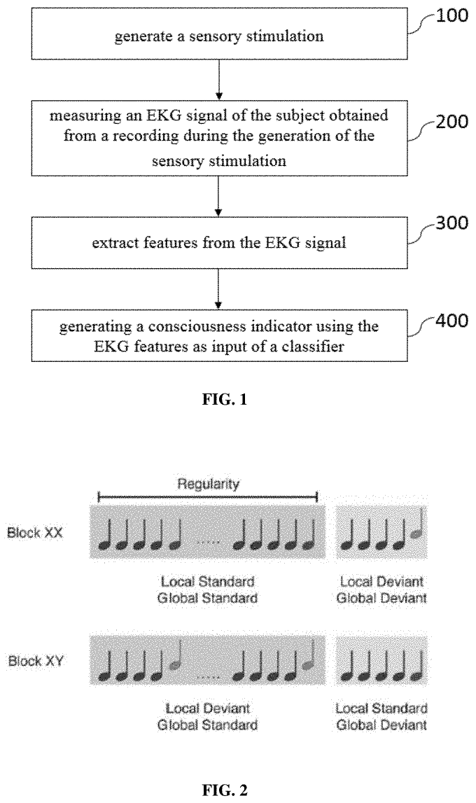

As shown in FIG. 1, the method of the present invention comprises four main steps, starting from the generation of a sensory stimulation 100. The second main step 200 consists in the measure of an electrocardiographic signal recorded from the subject during the sensory stimulation. The third step 300 extracts EKG features from the electrocardiographic signal while the last step 400 is configured to deduce a consciousness indicator from the classification of the EKG features.

The sensory stimulation 100 is configured to induce a cognitive process in the stimulated subject. The human brain has the ability to extract patterns or regularities in its environment, e.g. object A is always followed by object B but never by object C. The brain can detect transitional probabilities in an automatic way, i.e. even when the subject's attention is distracted, or when stimuli are presented below the threshold of awareness. Automatic brain responses to a violation of a rule (or regularity) can also be detected if the stimuli are in close or local temporal vicinity (i.e. within few seconds). Mismatch responses can be produced with complex sequences such as a melody or a rhythm even in unconscious subjects.

The sensory stimulation of the present invention may be configured so as to stimulate any of the five senses. Therefore, the sensory stimulation may comprise any stimuli among the auditory stimuli, visual stimuli, somatosensory stimuli, olfactory stimuli, gustatory stimuli, or a combination thereof.

According to one embodiment, the sensory stimulation comprises multiple consecutive stimuli including at least a first stimulus and a second stimulus relating to the same sense and different from one another.

According to one embodiment, the sensory stimulation comprises cross modality stimuli with a stream of stimulation of one sense (i.e. auditory sounds) which can be predictive of the stimulation occurring in another sense (i.e. visual stimuli).

According to one embodiment, the sensory stimulation comprises only auditory stimuli.

According to one embodiment, the sensory stimulation comprises multiple auditory trials having a predefined intertrial interval, each auditory trial formed by N consecutive auditory stimuli having a predefined time duration with a predefined gap between the auditory stimuli onsets. In this embodiment, the auditory stimulation has a first percentage of local standard trials comprising N identical auditory stimuli and a second percentage of locally deviant trials comprising the first N-1 identical auditory stimuli and the N^{th} auditory stimulus different from the preceding N-1 auditory stimuli, wherein N is equal or superior to 2

In one embodiment, the cognitive processing is prompted by means of auditory ‘local-global’ paradigm. This ‘local-global’ paradigm is based on an auditory oddball paradigm in which a sequence of sounds is presented at each auditory trial. The sequence is composed of a standard sound repeated a certain number of times, followed by a deviant sound. The comparison to a condition in which all the sounds of the sequence are standard typically reveals the occurrence of the mismatch negativity. Crucially, if the deviant sequence is highly frequent within a block, the subjects would expect the last sound to be deviant. The sequence is thus standard at the global level (i.e. over the experimental block) and deviant at the local level (i.e. within a single trial). The local standard sequence becomes a global deviant and triggers the occurrence of a P300 component. In sum, the ‘local-global’ paradigm allows orthogonal manipulations of automatic versus conscious brain responses to regularity violations.

According to the embodiment shown in FIG. 2, each auditory trial is formed by 5 consecutive sounds lasting for a predefined window ranging from a few milliseconds to hundreds of milliseconds, with a predefined gap between the auditory stimulus' onsets ranging from a few milliseconds to hundreds of milliseconds and an intertrial interval ranging from 1 to 2 milliseconds. The fifth auditory stimulus can be either equal to the first 4 (i.e. XXXXX) or different from the first 4 (i.e. XXXXY); which defines whether the trial is standard or deviant at a local (or within trial) level. The second level of regularities is defined across trials (or at a global level); frequent trials define the regularity, and rare ones violate this regularity. Frequent trial represents a percentage of the trials superior to 50% and rare trial represent remaining trials. Two types of stimulation blocks are played in the auditory stimulation: the XX blocks and XY blocks. In the XX blocks, the frequent stimulus corresponds to 5 equal auditory stimuli (local standard and global standard). In contrast, the infrequent stimulus corresponds to 4 equal auditory stimuli followed by a fifth different auditory stimulus (local deviant and global deviant). In the XY blocks, the frequent stimulus corresponds to 4 equal auditory stimuli and a fifth different auditory stimuli (local deviant and global standard), while the infrequent stimulus corresponds to 5 equal auditory stimuli (local standard and global deviant). The local effect is quantified by contrasting all local deviant trials versus all local standard trials. The global effect is quantified by contrasting all global deviant trials versus all global standard trials.

As a variant, the sensory stimulation may be a non-structured auditory stimulation, such a recited story in which semantical errors are included. In one example, the sensory stimulation is a child story associated with the emission of a simultaneous sequence of tones. In one exemplary embodiment, the sensory stimulation is a movie and in order to characterize the sensory stimuli associated to the movie the semantic content of the movie is studied by automatic text analysis of the subtitles (in English). The following procedure is implemented: (1) from the subtitle document extract timestamps and convert to elapsed seconds; (2) tokenize, lemmatize, remove non-English words and stop words; (3) for every word and time, obtain the semantic distance between that word and a set of predefined words using a pre-trained deep neural network; (4) compute the average semantic distance for all words in each time stamp; and (4) apply a sliding window smoothing (one minute). This procedure lead to time series of ‘semantic content’ for the proposed predefined words during the movie.

The sensory stimulation 100 is followed by the step 200 of measuring at least one electrocardiographic signal of the subject during the generation of the sensory stimulation.

In the embodiment of FIG. 2, the extraction step 300 is configured to extract at least one EKG feature from the electrocardiographic signal associated to cognitive processes to be used as input of a classifier to generate the consciousness indicator.

According to one embodiment, the extraction step 300 comprises a step of performing a delineation of the EKG electrocardiographic signal identifying for each heart beat a QRS complex, a T-wave and a P-wave. This delineation step may be performed with algorithm based on wavelet transform or classification method such as Support Vector machine, or a combination thereof.

According to one embodiment, the EKG features obtained from the electrocardiographic signal are chosen among the following:
heart rate,
heart rate variability in the high frequencies,
heart rate variability in the low frequencies,
heart rate variability in the very low frequencies,
cardiac modulations to the stimulation paradigm in the time interval between the onset of the N^{th} stimulus and the following R peak for a local effect, and
cardiac modulations to the stimulation paradigm in the time interval between the onset of the N^{th} stimulus and the following R peak for a global effect.

Parameters deriving from the shape of the T wave and P wave, such as the timing, the amplitude or the symmetry, may be further used as EKG features to be provided as input to the classifier.

According to one embodiment, at least one of the EKG features obtained from the electrocardiographic signal is the RT distance, being the distance between the R peak and the T peak which represents the repolarization of the ventricles. Indeed, EKG directly measured with electrocardiogram electrodes allow to detect T peak which is a low intensity peak.

According to one embodiment, the calculation of the heart rate is computed by averaging the differences between consecutive R peaks during the whole recording.

According to one embodiment, calculating heart rate variability for the electrocardiographic signal comprises the estimation of heart rate variability spectral variables. Such heart rate variability spectral variables which are obtained by computing the power spectrum decomposition on the point events time series from the detected R peaks. Power spectral density may be estimated in whole recording using Welch's method. In one example, the power spectral density is estimated in whole recording using Welch's method with 32.768 samples (131.072 seconds) per segment and 28.672 samples (114.688 seconds) overlap using a Hanning window. Heart rate variability variables are extracted from the sum of the spectral power in 3 frequency bands: very low frequency (i.e. range 50-0.04 Hz), low frequency (i.e. range 50.04-0.15 Hz) and high frequency (i.e. range 50.15-0.4 Hz).

According to one embodiment, the extraction step 300 further comprises the step of defining a time interval as the interval between the onset of at least one stimulus and the following R peak and extracting one EKG feature calculating a modulation of the correlation of the phase of the cardiac cycle and the timing of the sensory stimulation.

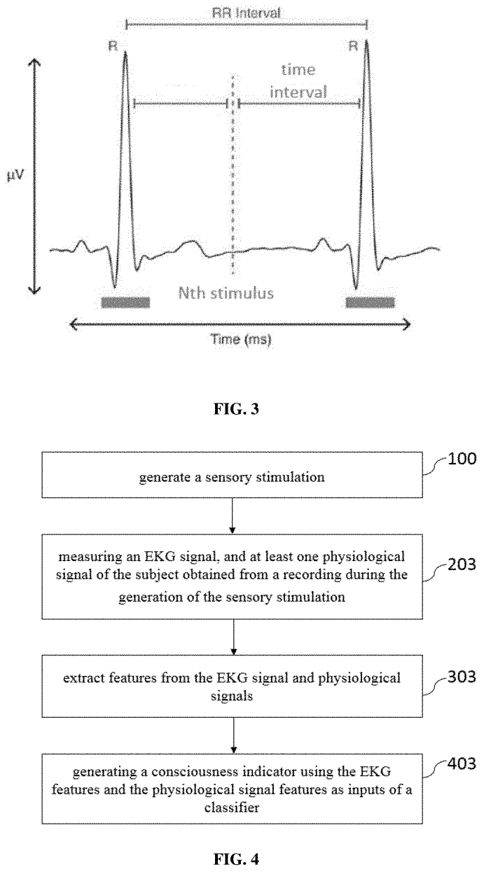

FIG. 3 shows two consecutives electrocardiographic QRS complexes defining an RR interval. This image shows as well the time interval between the onset of the N^{th} stimulus and the following R peak.

According to one embodiment, R peak onsets were obtained automatically by the algorithm described in Elgendi (Elgendi M., “Fast QRS detection with an optimized knowledgebased method: evaluation on 11 standard ECG databases.” PLoS One 2013;8:e73557).

According to the embodiment of FIG. 2, the deduction step 400 consists in the generating a consciousness indicator using the EKG features as input of a classifier.

According to a second embodiment represented in FIG. 4, the method of the present invention further comprises a step of receiving at least one additional physiological data of the subject recorded during the sensory stimulation 203. The physiological signal may be chosen among the following: respiratory activity measurements, electrodermal activity measurements, metabolic state measurements, pupillometry measurements.

According to this second embodiment, the extraction step is configured to further extract at least one physiological feature 303. Said physiological features may be calculated as a modulation of the correlation of the physiological signal and the timing of the sensory stimulation. According to one embodiment, the physiological features are used as further input of the classifier 403.

From the respiratory activity measurements, multiple features could be calculated as input for the classifier such as the phase shift (i.e. modulation of the correlation of the respiratory activity signal and the timing of the sensory stimulation), the amplitude, the shape and the like.

The feature extracted from the electrodermal activity measurements may be the amplitude of the response in correlation with the timing of the sensory stimulation.

Extraction of features from metabolic state measurements provides information concerning the emission of respiratory metabolic gases (i.e. CO₂, Acetone, Isoprene and the like).

Pupillometry measurements allow to extract features concerning the response in pupil contraction as a function of the timing o the sensory stimulation.

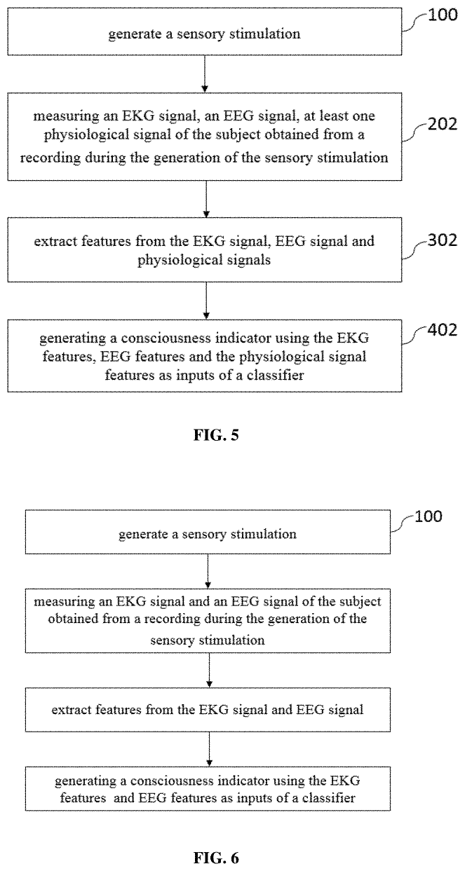

According to a third embodiment shown in FIG. 5, the method of the present invention comprises a step 202 of measuring an electroencephalographic signal of the subject during the generation of the sensory stimulation.

In this embodiment, the extraction step further comprises the extraction of at least one EEG feature from the electroencephalographic signal 302 so as to be a further input of the classifier for the generation of the consciousness indicator.

According the third embodiment, at least one of the following measures are performed on the electroencephalographic signal: permutation entropy, Kolmogorov Complexity, Weighted Symmetrical Mutual Information, Alpha PSD, Normalized Alpha PSD, Beta PSD, Normalized Beta PSD, Delta PSD, Normalized Delta PSD, Theta PSD, Normalized Theta PSD, Median Power Frequency, Spectral Entropy 90, Spectral Entropy 95, Spectral Entropy, Contingent Negative Variation, short-latency sensory potentials, mid-latency sensory potentials, late-latency sensory potentials, GD-GS full contrast (represent the ‘Global Effect’ and it corresponds to the contrast of all the Global deviant trials versus all the Global standard trials), LD-LS full contrast (‘local Effect’ using all blocks and it corresponds to the contrast of all the Local deviant trials versus all the Local standard trials), LSGD-LDGS full contrast (contrast of the rare trials versus the frequent trials), LSGS-LDGD full contrast (contrast of the frequent trials versus the rare trials), Contrasted P3a (Local Deviant vs Local Standard), Contrasted P3b (Global Deviant vs Global Standard), Contrasted MMN (Local Deviant vs Local Standard), Decoding of Local Deviant vs Local Standard and Decoding of Global Deviant vs Global Standard.

According to this embodiment, the EEG features are calculated as:
mean across epochs (times) and mean across sensors (EEG electrodes) of one of the above cited measure;
mean across epochs and standard deviation across sensors of one of the above cited measure;
standard deviation across epochs and mean across sensors of one of the above cited measure; and/or
standard deviation across epochs and standard deviation across sensors of one of the above cited measure.

According to one embodiment, the deduction step 402 consists in providing the EKG features, the EEG features and physiological features as input of the classifier configured to generate the consciousness indicator.

According to one embodiment, the classifier is a previously trained machine learning technique.

According to one embodiment, the classifier is a Support Vector Machine. In machine learning, support vector machines (SVMs) are discriminative classifiers formally defined by a separating hyperplane. The Support Vector machine of the present method may be configured to perform linear classification or non-linear classification using the pattern analysis. Indeed, in order to analyze the relevance and independence of the markers regarding the diagnosis of subjects with disorders of consciousness, multivariate pattern analysis (MVPA) could be used in combination with wrappers algorithms for the selection of pertinent markers as features for the classifier. The multivariate pattern analysis method consists in training classifiers with different sets of features and comparing the obtained performance. Based on the performance comparisons, a set of features can be defined as (1) strongly or weakly relevant, when they are partially independent and contribute to an optimal classification; or (2) irrelevant, when they do not contribute to the classification.

In one example, the multivariate pattern analysis method is done using 120 EEG-extracted markers (corresponding to quantification of power spectrum and complexity in individual EEG sensors and information sharing between EEG sensors) and 8 EKG-extracted markers. In one example, the kernel function of the Support Vector Machine selected to suit the problem of the present invention is a sigmoid kernel. This Support Vector Classifier is trained upfront to distinguish between two main classes of patient with disorders of consciousness: vegetative state/unresponsive wakefulness syndrome (VS/UWS) and minimally conscious state (MCS). A penalization parameter equal to 1 may be used.

According to one embodiment, prior to the training of the classifier, relevant EKG features and/or EEG features and/or physiological features are automatically selected keeping the highest 20% of the ANOVA F scores. For the supervised training of the Support Vector Classifier the labelled EEG data based on the Coma Recovery Scale-Revised scores (a behavioral scale for the diagnosis of disorders of consciousness) may be used.

Support Vector Machine is one of the most popular techniques of machine learning. Other techniques such as random forest may be implemented in the method of the present invention.

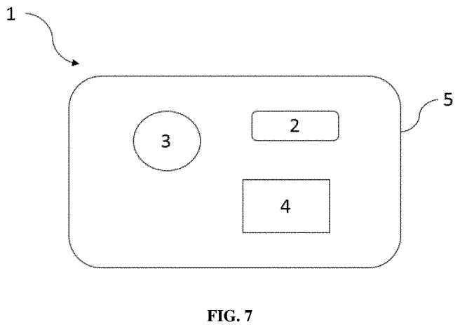

According to a fourth embodiment represented in FIG. 6, the method of the present invention uses only EKG features and EEG features as input of the classifier to deduce the consciousness indicator.

According to one embodiment, the consciousness indicator is compared to a threshold.

The present invention further relates to a system 1 for the generation of a consciousness indicator for a non-communicating subject being sensorially stimulated. The main components of the system 1 are an acquisition module 3 and a calculation module 4.

According to one embodiment, the system 1 comprises an acquisition module 3 configured to perform the acquisition of an electrocardiographic signal during the sensory stimulation of the patient. The system 1 may comprise one or more acquisition devices such as an electrocardiogram, a high density electroencephalogram, a respiratory belt, a microphone, an eye tracking device, a camera, a temperature sensor, a pressure sensor, a CO₂ sensor, a volatile organic compound sensor, a nasal cannula, and the like. Or alternatively, the system 1 may be configured to control said acquisition devices. The system 1 may further comprise a communication module configured to allow the communication between the acquisition module and the acquisition device to control signal acquisition and retrieve data. The communication module may be equipped to perform wireless data transfer.

According to one embodiment, the calculation module 4 is configured to extract at least one feature from the electrocardiographic signal and deduce a consciousness indicator using the EKG features as input of a classifier according to the embodiment described hereabove. The calculation module 4 may be further configured to extract features from electroencephalographic signal and/or physiological signal and use electroencephalographic signal and/or physiological signal as further input for the classifier in order to deduce the consciousness indicator.

According to one embodiment, the system 1 further comprises a stimuli generator 2 configured to generate a sensory stimulation according to the embodiment described here above.

According to one embodiment, at least the acquisition module 3 and the calculation module 4 are comprised in a same portable element 5 configured to locally implement the method according to any one of the embodiments described hereabove. This feature allows the user to take the whole system directly in the hospital facility where the subject is staying and to perform on site both acquisition and data analysis.

FIG. 7 shows an embodiment of the present invention wherein the portable element 5 further comprises the stimuli generator 2.

The steps of the method implemented by the different modules described herein are implemented by hardware components. Examples of hardware components include controllers, sensors, generators, drivers, memories, comparators, arithmetic logic units, adders, subtractors, multipliers, dividers, integrators, neural networks, signal separators, calculators, extractors, determiners, and any other electronic components known to one of ordinary skill in the art. In one example, the hardware components are implemented by computing hardware, for example, by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the steps described herein. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The present invention further relates to a program comprising instructions which, when the program is executed by a computer, cause the computer to carry out the steps of the method according to any one of the embodiments described here above.

Instructions of the program, to perform the methods as described above, are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

Yet another aspect of the present invention relates to a computer-readable storage medium comprising instructions which, when the program is executed by a computer, cause the computer to carry out the steps of the computer-implemented method according to anyone of the embodiments described here above. According to one embodiment, the computer-readable storage medium is a non-transitory computer-readable storage medium.

Computer programs implementing the method of the present embodiments can commonly be distributed to users on a distribution computer-readable storage medium such as, but not limited to, an SD card, an external storage device, a microchip, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While various embodiments have been described and illustrated, the detailed description is not to be construed as being limited hereto. Various modifications can be made to the embodiments by those skilled in the art without departing from the true spirit and scope of the disclosure as defined by the claims.

1-15. (canceled)

16. A method for the generation of a consciousness indicator for a non-communicating subject, comprising:
generating a sensory stimulation involving at least one of the five senses, said sensory stimulation comprising multiple consecutive stimuli including at least a first stimulus and a second stimulus relating to the same sense and different from one another;
measuring an electrocardiographic signal of the subject during the generation of the sensory stimulation;
extracting at least one EKG feature from the electrocardiographic signal associated to cognitive processes; and
generating a consciousness indicator using the EKG features as input of a classifier.

17. The method according to claim 16, wherein the stimuli of the sensory stimulation are auditory stimuli, visual stimuli, somatosensory stimuli, olfactory stimuli, gustatory stimuli, or a combination thereof.

18. The method according to claim 16, wherein the extraction at least one EKG feature from the electrocardiographic signal comprises:
performing a delineation of the EKG electrocardiographic signal identifying for each heart beat a QRS complex, a T-wave and a P-wave;
calculating the heart rate and heart rate variability for the electrocardiographic signal from the delineation of the EKG electrocardiographic signal;
defining a time interval as the interval between the onset of at least one stimulus and the following R peak and extracting one EKG feature calculating a modulation of the correlation of the phase of the cardiac cycle and the timing of the sensory stimulation.

19. The method according to claim 18, further comprising receiving at least one additional physiological signal of the subject recorded during the sensory stimulation chosen among the following: respiratory activity measurements, electrodermal activity measurements, metabolic parameter measurements, pupillometry measurements.

20. The method according to claim 19, further comprising extracting at least one physiological feature by calculating a phase shift of the physiological signal.

21. The method according to claim 20, wherein the at least one physiological feature are used as further input of the classifier.

22. The method according to 16, further comprising measuring an electroencephalographic signal of the subject during the generation of the sensory stimulation.

23. The method according to claim 22, further comprising:
extracting at least one EEG feature from the electroencephalographic signal; and
using the EEG features as further input of the classifier to generate a consciousness indicator.

24. The method according to claim 16, wherein the classifier is a previously trained machine learning technique.

25. The method according to claim 16, further comprising a comparison of the consciousness indicator to a predefined threshold.

26. A non-transitory computer readable medium comprising instructions which, when the program is executed by a computer, cause the computer to carry out the method according to claim 16.

27. A system for the generation of a consciousness indicator for a non-communicating subject undergoing sensory stimulation, comprising:
at least one processor configured for:
acquiring at least an electrocardiographic signal of the subject during the generation of the sensory stimulation, said sensory stimulation involving at least one of the five senses; and
extracting at least one EKG feature from the electrocardiographic signal and generating a consciousness indicator using the EKG features as input of a classifier.

28. The system according to claim 27, further comprising a stimuli generator configured to generate sensory stimulation, said sensory stimulation comprising multiple consecutive stimuli including at least a first stimulus and a second stimulus relating to the same sense and different from one another.

29. The system according to claim 27, wherein at least the at least one processor is comprised in a same portable element configured to locally implement a method for the generation of a consciousness indicator for a non-communicating subject, comprising:
generating a sensory stimulation involving at least one of the five senses, said sensory stimulation comprising multiple consecutive stimuli including at least a first stimulus and a second stimulus relating to the same sense and different from one another;
measuring an electrocardiographic signal of the subject during the generation of the sensory stimulation;
extracting at least one EKG feature from the electrocardiographic signal associated to cognitive processes; and
generating a consciousness indicator using the EKG features as input of a classifier.

30. The system according to claim 27, wherein the at least one processor is further configured to:
performing a delineation of the EKG electrocardiographic signal identifying for each heart beat a QRS complex, a T-wave and a P-wave;
calculating the heart rate and heart rate variability for the electrocardiographic signal from the delineation of the EKG electrocardiographic signal;
defining a time interval as the interval between the onset of at least one stimulus and the following R peak and extracting one EKG feature calculating a modulation of the correlation of the phase of the cardiac cycle and the timing of the sensory stimulation.

31. The system according to claim 27, further comprising at least one input adapted to receive at least one additional physiological signal of the subject recorded during the sensory stimulation chosen among the following: respiratory activity measurements, electrodermal activity measurements, metabolic parameter measurements, pupillometry measurements.

32. The system according to claim 31, wherein the processor is further configured to extract at least one physiological feature using the physiological signal and provide said physiological feature as input of the classifier.

33. The system according to claim 27, wherein the processor is further configured to compare the consciousness indicator to a predefined threshold.
